(19) United States
(12) Patent Application Publication
(10) Pub. No.: US 2024/0188942 A1
(43) Pub. Date: Jun. 13, 2024

(54) SAMPLE COLLECTION DEVICES, SYSTEMS, AND METHODS

(71) Applicant: SPECTRUM SOLUTIONS, LLC, Draper, UT (US)

(72) Inventors: Neil Jeremy JOHNSON, Riverton, UT (US); Mark Martin JENSEN, Draper, UT (US)

(21) Appl. No.: 18/532,837

(22) Filed: Dec. 7, 2023

Related U.S. Application Data
(60) Provisional application No. 63/524,324, filed on Jun. 30, 2023.
(60) Provisional application No. 63/465,725, filed on May 11, 2023.
(60) Provisional application No. 63/386,618, filed on Dec. 8, 2022.

(51) Int. Cl. A61B 10/00; B01L 3/00

(52) U.S. Cl. CPC A61B 10/0051; A61B 10/0096; B01L 3/5029; B01L 2400/06

(57) ABSTRACT

A biological sample collection device (e.g., swab) can include elongated body (e.g., shaft) extending from a collecting portion (e.g., swab body or tip) at a first end to a connecting portion at a second end along a longitudinal axis. The elongated shaft and the swab body can be formed of flexible materials. The connecting portion may be configured to associate with a handle or sealing cap. A biological sample collection system can include the sample collection device, a sealing cap, and a sample preservation vessel. In use, the sample collection device may be associated with the sealing cap, used at a biological sample site to collect the biological sample, and enclosed in the sample preservation vessel by associating the sealing cap with the sample preservation vessel.

### CROSS REFERENCE TO RELATED APPLICATION

This application claims the benefit of U.S. Provisional Application No. 63/524,324, filed Jun. 30, 2023, U.S. Provisional Application No. 63/465,725, filed May 11, 2023, and U.S. Provisional Application No. 63/386,618, filed Dec. 8, 2022, which are incorporated by reference in their entirety.

### BACKGROUND

### Technical Field

This disclosure generally relates to devices, systems and methods for collecting and storing biological samples. More specifically, the present disclosure relates to devices, systems, and methods that include a sample collection swab and preservation vessel for collecting and preserving biological samples for later testing in a laboratory or other biological sample analysis facility.

### Background and Relevant Art

Field collection of biological samples can provide scientists, physicians, geneticists, epidemiologists, and similar personnel with invaluable information. For example, access to a fresh sample of a subject's blood, purulent discharge, or sputum can help a physician or epidemiologist to isolate or identify a causative agent of infection. Similarly, a saliva sample can permit a scientist or geneticist access to the requisite nucleic acid for genetic sequencing, phylotyping, and other genetic-based studies. In the foregoing examples, in addition to many other situations, it is desirable to work with a fresh biological sample to ensure accurate results. However, isolation of the probative composition (e.g., nucleic acid, proteins, chemicals, etc.) often requires the use of specialized equipment and often benefits from controlled laboratory conditions.

It can be inconvenient and sometimes improbable to require patients/individuals to travel to a biological sample collection center having the appropriate equipment and desirable controlled environment for sample preparation. Similarly, it may be difficult for personnel to directly access the patient/individual, particularly if the sample size is large and/or geographically diverse (e.g., as can be found in large genetic studies of thousands of individuals across an entire country, ethnic population, or geographic region). Further complicating this issue, it is often beneficial to immediately process any procured biological sample, and field personnel may be limited by lack of access to appropriate specialized equipment or to a controlled environment for high-fidelity sample processing.

Some biological sample collection devices and kits have addressed some of the foregoing issues. For example, some commercial kits provide a user with a vial for receiving a biological sample and a preservation reagent that can be added to the collected biological sample, acting to preserve elements within the biological sample (to a certain extent and for a period of time). However, implementations of self-collection systems often rely on inexperienced or untrained individuals to collect and deposit the biological sample into the receiving vessel. This presents a number of problems, including, for example, technical training and precise measurements often required to properly collect and preserve the biological sample for later processing. In the absence of such, it is important to provide a biological sample collection system that can be easily implemented by a novice user and which can preserve the received biological sample for later processing.

Accordingly, there are a number of disadvantages with biological sample collection and preservations systems that can be addressed.

### BRIEF SUMMARY

Implementations of the present disclosure solve one or more of the foregoing or other problems in the art with apparatuses, systems and methods for collecting and preserving a biological sample. In particular, one or more implementations can include a sample collection device—or a system including the same—for collecting and preserving a biological sample. The device (e.g., swab) can include an elongated body (e.g., shaft) extending from a first end to a second end along a longitudinal axis, and a collecting portion (e.g., a swab body or tip) at the first end of the elongated shaft including a plurality of protrusions. The elongated shaft and the collecting portion or swab body may each be configured to be flexible, such as in order to capture viral and/or bacterial cells of interest in mucus without scraping cells from mucous membranes of a subject. The swab body or tip can also include a plurality of flexible protrusions extending outwardly therefrom, the protrusions defining channels in the swab body or tip for collecting a biological sample.

In some implementations, the second end of the elongated shaft can include a connecting portion configured to associate with a handle. The handle can comprise a sealing cap configured to associate with a sample collection and preservation vessel and may include a reagent chamber for storing a preservation reagent.

The present disclosure also includes biological sample collection systems—or kits including the same—for collecting and preserving a biological sample. In some embodiments, a biological sample collection system includes a sample collection device (e.g., swab) having an elongated body (e.g., shaft) extending along a longitudinal axis from a collecting portion (e.g., swab body or tip) at a first end to a connecting portion at a second end, and a sample preservation vessel having an opening for receiving the sample collection swab and associated biological sample. The biological sample collection system can additionally include a sealing cap configured to associate with the connecting portion of the sample collection swab and with the sample preservation vessel. In some embodiments, the sealing cap can include a reagent chamber for storing a measure of preservation reagent and a valve that can be selectively opened to release the preservation reagent into the sample preservation vessel. Associating the sealing cap with the sample preservation vessel can cause a physical rearrangement of the valve components, thereby permitting the preservation reagent to pass from the regent chamber into the sample preservation vessel.

The present disclosure also includes methods for collecting and preserving a biological sample. An exemplary method includes providing a disclosed sample collection device or swab to a subject for capturing a biological sample in a collecting portion (e.g., swab body) of the sample collection device, collecting a biological sample on the swab body, inserting the sample collection device into the sample preservation vessel, and associating a sealing cap with the sample preservation vessel, for example, to open a valve and release a preservation reagent held in a reagent chamber in the sealing cap into the sample preservation vessel.

Accordingly, devices, systems, methods, and kits for collecting a biological sample are disclosed herein. This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 is a cross-sectional view of a sample collection device/swab including an elongated body/shaft having a biological sample collecting portion (swab body) attached thereto.

FIG. 2 is a cross sectional view of the elongated body/shaft shown in FIG. 1.

FIG. 3 is a perspective view of the collecting portion (swab body) shown in FIG. 1.

FIG. 4 is an exploded, cross sectional side view of a multi-piece, self-contained sample collection system or kit incorporating the sample collection swab shown in FIG. 1.

FIG. 5A is a cross sectional view of the sample collection system or kit shown in FIG. 4 with the valve thereof in a closed position and preservation reagent in a reagent chamber of the sealing cap.

FIG. 5B is a cross sectional view of the sample collection system or kit shown in FIG. 5A with the valve thereof in an open position and preservation reagent released from the reagent chamber into the sample preservation vessel.

FIGS. 6A and 6B are top and bottom perspective views of a core of the valve shown in FIGS. 5A-5B.

FIGS. 7A and 7B are top and bottom perspective views of a collar of the valve shown in FIGS. 5A-5B.

FIG. 8A is a cross sectional view of retaining elements of the elongated body/shaft shown in FIG. 2 taken along line 8A-8A.

FIG. 8B is a bottom view of the valve shown in FIG. 4;

FIG. 8C is the bottom view of the valve shown in FIG. 8B having the retaining elements of FIG. 8A coupled thereto.

FIG. 9 is an alternative embodiment of sample collection system shown in FIG. 4 having an alternative sample collecting portion (swab body).

FIG. 10 is an enlarged perspective view of the collecting portion (swab body) shown in FIG. 9.

FIGS. 11A and 11B illustrate alternative embodiments of sample collection systems having alternative sample preservation vessels and alternative sealing caps.

FIG. 12A is an alternative embodiment of sample collection system shown in FIG. 4 having an alternative sample collecting portion (swab body).

FIG. 12B is a plan view of the side face of the collecting portion (swab body) shown in FIG. 12A.

FIG. 12C is an elevation view of the end face of the collecting portion (swab body) shown in FIG. 12A.

FIG. 12D is a bottom plan view of the collecting portion (swab body) shown in FIG. 12A.

FIG. 13A is a perspective view of an alternative embodiment of the collecting portion (swab body) shown in FIGS. 12A-12D;

FIG. 13B is an elevation view of the end face of the collecting portion (swab body) shown in FIG. 13A;

FIG. 14A is a chart that graphically illustrates the inter feature spacing of different swabs bodies made from different types materials; and

FIG. 14B is a chart that graphically illustrates the results of a surface spread test, which demonstrates how dilution of saliva with a preservation reagent reduced surface tension and assists in releasing a saliva sample from the swab made from a certain type of material.

### DETAILED DESCRIPTION

Embodiments of the present disclosure address one or more problems in the art of devices, systems, kits, and/or methods for collecting and preserving a biological sample. A biological sample can be collected and its contents evaluated for various reasons, including, for example, identifying or characterizing a causative agent of disease (e.g., for treatment of the affected individual, for epidemiological reasons, etc.) or for genetic analysis of a subject's nucleic acid (e.g., genetic phylotyping, gene expression studies, genome sequencing, etc.). In most instances, including within the foregoing examples, it is desirous that the fidelity of the biological sample be maintained so that it retains its probative value. However, collecting and preparing biological samples for analysis has traditionally been a complex procedure performed by a skilled technician or specialized professional. This is problematic for obvious reasons, including the time and cost associated with individually collecting and transporting biological samples, particularly when subjects reside in disparate locations and require service from personnel with the required skills to properly collect and preserve the biological sample.

Various types of devices may be used to collect biological samples. In one aspect, a “swab” device may be used having a shaft and a swab tip or body, where a user controls the swab using the shaft thereof to apply the swab tip or body to a subject for collecting a sample therefrom. In many instances, at least the swab tip or body of a swab device may comprise an absorbent or non-absorbent material for collecting the sample. For example, a swab body of a nasopharyngeal swab may include a wadded cotton or similar material connected to a wood, rolled paper, or plastic shaft. Alternatively, the swab body may comprise a molded polymer, such as an elastomer, with ribs and/or indentations or recesses that can collect a biological sample from a person's mouth or other mucous membrane without scraping off human cells. The nasopharyngeal swab may be inserted to a subject's nostril, mouth, or throat to collect a sample from the surface of the mucous membranes for evaluating a suspected viral infection. Various other types of swabs or similar devices may be configured for different diagnostic tests and/or collecting samples from other anatomical sites of a subject.

A “biological sample” is a target material from a subject that can be used for diagnostic, prognostic, genetic, or other scientific analysis. For example, a biological sample may comprise a secretory fluid (whether host or pathogen related) from a patient. This can include, for example, a non-human cell sample that includes any of a bacterium, virus, protozoa, fungus, parasite, and/or other prokaryotic or eukaryotic symbiont, pathogen, or environmental organism. The term “biological sample” is also understood to include fluid samples such as blood, urine, saliva, and cerebrospinal fluid and extends to other biological samples including, for example, mucus from the nasopharyngeal region and the lower respiratory tract (i.e., sputum).

As used herein, the “probative component” of the biological sample can refer to any protein, nucleic acid, surface moiety, or other compound that can be isolated from the biological sample. The probative component may be or include nucleic acid, such as DNA and RNA.

Accordingly, it may be desired in some embodiments to obtain a biological sample that does not include cells of a patient's mucous membranes, skin, or other epithelial tissue, such as may result from a tip portion or shaft of a swab scraping a patient's epithelial tissue in the nose, mouth and/or throat. For example, it may be desired to obtain a biological sample for identifying the presence of a virus, bacterium, or fungus, where the presence of genetic material of the patient's skin or membranes may confound or impede detection. In other embodiments, it may be desired to obtain a biological sample that does not include material of the swab therewith. The exclusion or reduction of swab materials and/or the cells of a patient's epithelial tissue from a biological sample commonly requires preliminary processing of the biological sample to separate undesired components, such as by centrifugation or another process, prior to a diagnostic test or other intended use. It is desired to minimize and/or avoid such preliminary processing, advantageously reducing the expense and time required for analyzing a biological sample.

Embodiments provide sample collection and preservation devices, systems, and kits, and methods for using the same, which address one or more of the foregoing problems. For example, utilizing devices, systems, kits, and methods for collecting and preserving biological samples, as disclosed herein, removes the need of specialized personnel when collecting and initially preserving a biological sample. Furthermore, the disclosed embodiments simplify sample collection and preservation, which decreases the likelihood that even unskilled users will err when collecting and preserving biological samples.

As an illustrative example of the foregoing, sample collection devices (e.g., swabs) disclosed herein include an elongated body or shaft extending from a first end to a second end along a longitudinal axis.

A collection portion (e.g., swab body or tip) at the first end of the elongated body (e.g., shaft) may include a plurality of protrusions extending laterally relative to the longitudinal axis of the shaft and/or swab body, which can define channels or spaces therebetween. When used, the protrusions may act to guide the receipt of a biological sample from a subject into the channels or spaces in the collecting portion (swab body). The channels or recesses can also make it easier for a user to deposit or otherwise utilize a biological sample from the collecting portion. For example, a reagent may be applied to the collecting portion for removing and/or preserving the biological sample. The reagent may be drawn into the spaces of the swab body, such as by capillary action and, as the reagent is drawn into the spaces, the biological sample may be released from the spaces or otherwise mixed with the reagent.

A connecting portion at the second end of the elongated shaft may be configured to associate with a handle and/or a sealing cap, such as a sealing cap configured to associate with a sample preservation vessel. When used, the connecting portion may act to associate with the sealing cap, such that the sealing cap may be used as a handle for facilitating manipulation of the sample collection device. The connecting portion can also make it easier for a user to deposit a biological sample to a corresponding sample preservation vessel. For example, a sealing cap may be associated with the connecting portion for manipulating the elongated shaft to provide the swab body to a biological sample of a user. The swab body with the biological sample may be inserted into a sample preservation vessel corresponding to the sealing cap, and the sealing cap associating (e.g., by threaded engagement) with the sample preservation vessel for enclosing the sample collection device therein.

With respect to embodiments having a connecting portion, the connecting portion may be shaped to mechanically and rigidly interlock (e.g., via a friction fit or snap fit) with a handle or sealing cap such that the elongated body moves in unison with the handle or sealing cap. The connecting portion can include one or more retaining elements that can be sized and shaped to associate with a handle or a sealing cap. For example, the connecting portion can include opposing retaining elements configured to elastically compress against a base of a handle or sealing cap therebetween. In varying aspects, a retaining element can be annular and elastically fit an interior surface of a handle or sealing cap or otherwise be shaped to associate with a recess or element of the handle or sealing cap, forming a tight connection (e.g., via a friction fit or snap fit) therebetween. It should be appreciated that in some embodiments, an adhesive or similar material may be provided for securing the elongated body and/or the connecting portion to a handle or sealing cap.

As described in more detail below, the elongated shaft and the swab body of the sample collection device may each be formed from a flexible, non-absorbent material to facilitate capture of a target biological sample without scraping additional material from a surface or membrane on which the target biological sample is located. In various embodiments, the swab body may be formed from a material having a greater flexibility than a material of the elongated shaft.

Biological sample collection kits and systems disclosed herein may include at least a three-piece sample collection and preservation system. One aspect includes a sample collection device (e.g., swab), which can be detachably associated with a sealing cap of a sample preservation vessel. When used, the sealing cap may act as a handle to guide the collection of a biological sample from a user into a collecting portion (e.g., swab body or tip) of the sample collection device. The sealing cap can also make it easier for a user to deposit a biological sample into the sample preservation vessel. After inserting the sample collection device containing a biological sample into the sample preservation vessel, a user can associate the sealing cap with the sample preservation vessel. A reagent chamber in the sample preservation vessel or the sealing cap may be pre-filled with a predetermined amount of preservation reagent(s), and as the sealing cap is associated with the sample preservation vessel to seal the received biological sample within a sample preservation chamber, a valve is opened and the reagent(s) released from the reagent chamber and guided into the sample preservation vessel, mixing with and preserving the received biological sample from the swab body of the sample collection device.

As can be appreciated from the foregoing, in addition to alternative and/or additional embodiments provided herein, the devices, systems, kits, and methods of the present disclosure can be used by skilled or unskilled individuals with reduced likelihood of error associated with collecting and at least initially preserving a biological sample. Accordingly, implementations of the present disclosure can reduce the cost associated with procuring biological samples for diagnostic, scientific, or other purposes and can increase the geographic reach of potential sample collection areas without the need of establishing the necessary infrastructure (e.g., controlled environments conducive to sample collection and preservation, skilled personnel to physically collect, transport, and/or preserve the biological samples, etc.).

### Sample Collection Devices/Swabs

In one embodiment, a sample collection device (e.g., swab) is provided that may generally include an elongated body (e.g., shaft) defining a longitudinal axis of the device, the elongated body/shaft extending from a first end to a second end along the longitudinal axis. A collecting portion (e.g., swab body or tip) may be provided at the first end of the elongated shaft, which may be secured to or integrally formed with the elongated shaft. In some embodiments, protrusions may be formed in the swab body, which define spaces or recesses therebetween for collecting a biological sample. A connecting portion may be provided at the second end of the elongated shaft, the connecting portion configured to associate with a handle or sealing cap.

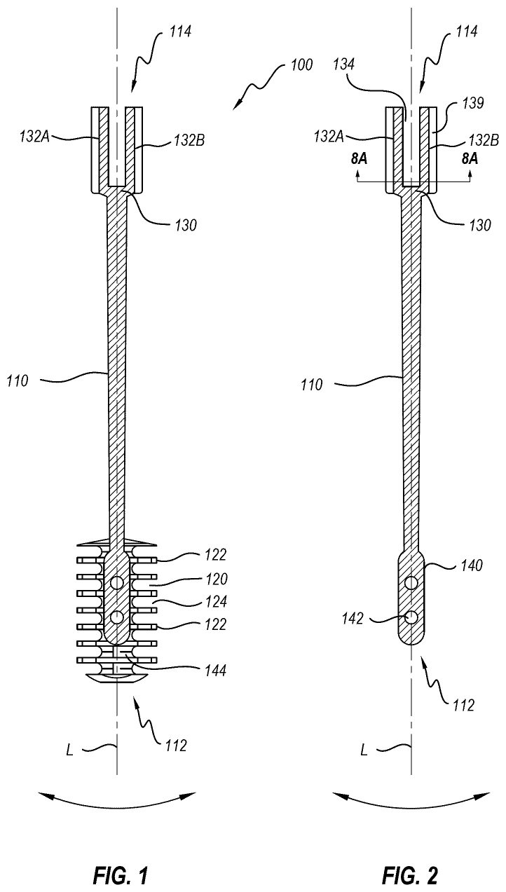

FIG. 1 is a cross-sectional view of a sample collection device (or swab) 100 that includes an elongated body (or shaft) 110 and a collecting portion (or swab body or tip) 120 disposed thereon. As shown in FIGS. 1 and 2, the elongated shaft 110 has an elongated, linear shape with a first end 112 and a second end 114 positioned opposite each other along a longitudinal axis L of the sample collection device 100. In some embodiments, the sample collection device 100 may be configured for use as a nasopharyngeal swab to be advanced through a subject's nostril to collect a sample from the surface of the respiratory mucosa for evaluating a suspected viral infection. In other embodiments, sample collection device 100 may be configured for use in collecting a saliva specimen from a mouth (e.g., via cheek swab) or throat of a patient. Various other configurations of and uses for the sample collection device 100 may be envisioned by those of ordinary skill in the art for accommodating insertion into other anatomical sites of a subject to reach different target locations (e.g., vagina or anus) and collect samples therefrom for different diagnostic tests.

As shown, the elongated shaft 110 of the sample collection device 100 may have a thickness that reduces or tapers from the second end 114 to the first end 112. This may increase flexibility of the elongated shaft 110 nearer the first end 112. The elongated shaft 110 may be provided with various shapes and profiles. For example, the elongated shaft 110 can be formed having a transverse cross section that is circular, elliptical, polygonal, irregular, or have other configurations. In addition, elongated shaft 110 can have a continuous thickness, one or more planar sides, one or more longitudinal grooves, etc., such as for achieving a desired profile or for ease of manufacture. In one embodiment, the elongated shaft 110 has a length extending between first end 112 and second end 113 that is at least or less than 4, 5, 6, 7, 8, 10, 12, 14, or 16 cm or is in a range between any two of the foregoing values. Other dimensions can also be used. In part, the length of elongated shaft 110 may vary depending on the intended use of sample collection device 100.

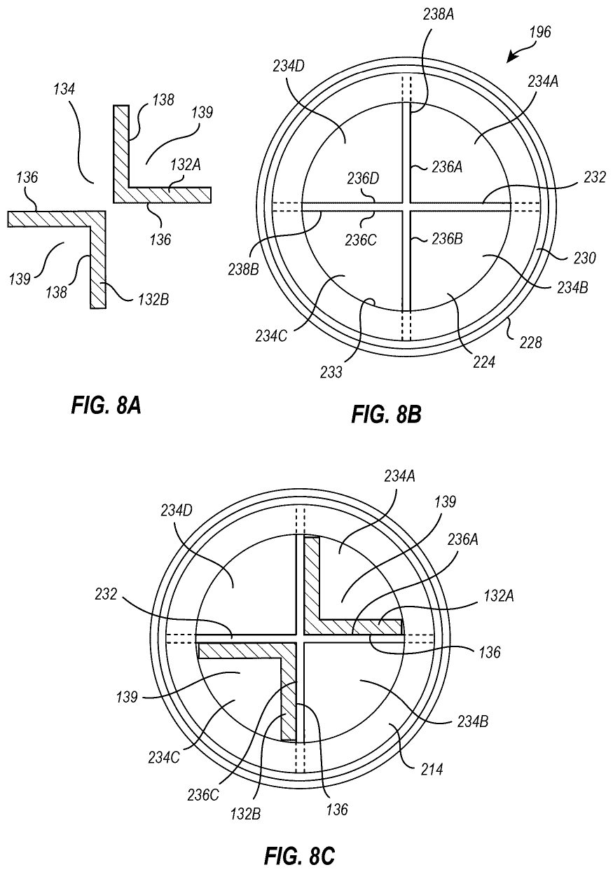

The second end 114 of the elongated shaft 110 may include a connecting portion 130 configured for connecting to a handle. In one embodiment, as discussed below in detail, the handle can be in the form of a sealing cap of a sample preservation vessel. In the depicted embodiment, the connecting portion 130 comprises a forked pair of opposing retaining elements 132A and 132B, which are configured to couple with a handle or sealing cap in a friction or snap fit engagement, as discussed below. Retaining elements 132A and 132B are spaced apart so as to bound a slot 134 therebetween and are typically disposed in parallel alignment. As depicted in FIG. 8A, in one embodiment, each retaining element 132A and 132B can have an L-shaped transverse cross section and include an inside face 136, between which slot 134 is bounded, and an opposing outside face 138. Each outside face 138 partially bounds a groove 139 extending along each retaining element 132A and 132B. The operation and alternatives to retaining elements 132A and 132B will be discussed below in greater detail.

In various embodiments, the connecting portion 130 may include one, two, three, four, or more retaining elements shaped to elastically fit an interior surface of a sealing cap or otherwise be shaped to associate with a recess or element of the sealing cap, forming a tight connection (e.g., via a friction fit) therebetween. It should be appreciated that in some embodiments, an adhesive or similar material connection may be provided for securing the elongated body and/or the connecting portion to a handle or a sealing cap, such as by melting or bonding.

Returning to FIGS. 1 and 2, embodiments of the sample collection device 100 may be configured to elastically deform from a linear shape when used for collecting biological samples from a subject. In this respect, the elongated shaft 110 may be formed from a flexible (e.g., polymer) material that is configured to bend away from the longitudinal axis L in response to a predetermined pressure. Advantageously, embodiments of the current disclosure may be configured with the elongated shaft 110 having a stiffness sufficient for insertion to an anatomical site and collection of a biological sample while retaining sufficient flexibility to prevent harm to a subject and/or unintended collection of epithelial cells from the subject, even when manipulated by an inexperienced user. Preferably, the elongated shaft 110 is formed of a polymer material having a Shore A scale durometer hardness of between 50 A to 90 A, more preferably between 60 A to 80 A. Other values can also be used.

While known swabs and related instruments have been employed for the collection of biological samples with some general success, prior art devices have a strong tendency to scrape the fragile epithelial cells of a collection site, such as in the nose, mouth, or throat. This leads to epithelial cells being stripped off and collected with the target biological sample, which then must be accounted for when evaluating the sample, either by separating out the undesired material or otherwise amplifying the target material for detection. By particular configuration of the stiffness of the elongated shaft 110, the collection of unintended materials such as from scraping a subject's epithelial cells can be reduced, minimized, or eliminated, even when operated by an inexperienced user. Examples of a flexible material of the elongated shaft may include a thermoplastic polymer, such as a medical grade polypropylene, polyethylene, other polyolefin, or the like.

The first end 112 may have a shape or thickness configured for receiving and/or securing to the collecting swab body 120. In the depicted embodiment, the first end 112 has an enlarged, elongated head 140 formed thereat. Head 140 can optionally have one or more holes 142 extending therethrough. In this configuration, if swab body 120 is overmolded onto head 140, such as through an injection molding process, the enlarged size of head 140 assists in retaining swab body 120 on first end 112 of the shaft 110. Furthermore, portions of the material used to form swab body 120 can flow through and solidify within holes 142 to further secure swab body 120 on first end 112. In alternative embodiments, swab body 120 need not be overmolded onto first end 112. Rather, first end 112 can be secured to swab body 120 using conventional coupling techniques, such as press fit connection, barbed connection, adhesive, welding, or other conventional mating techniques. The shape and size of first end 112 of elongated shaft 110 can vary depending on the type of connection being used.

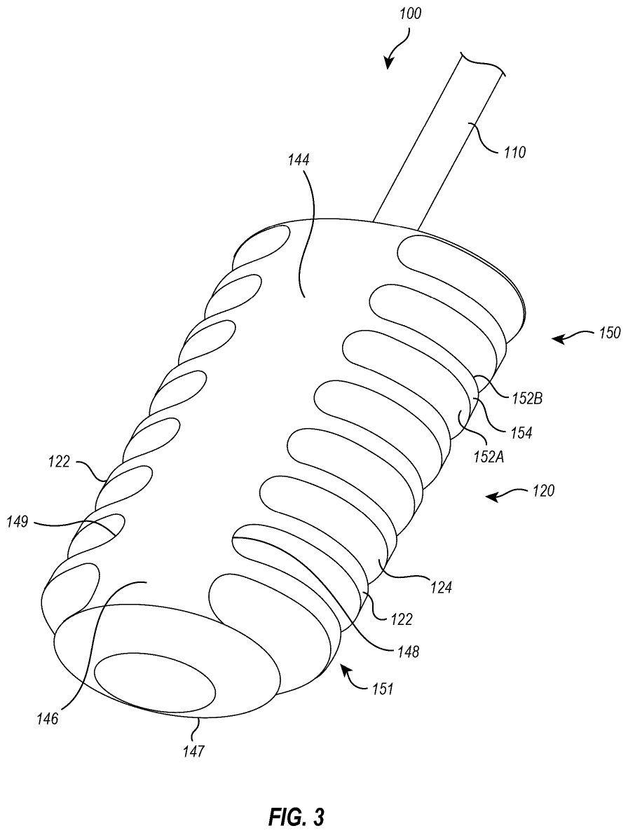

As depicted in FIGS. 1 and 3, the swab body 120 may comprise a plurality of protrusions 122 extending outward from the elongated shaft 110, the protrusions 122 defining channels or spaces 124 in the swab body 120 for receiving a biological sample therein. The channels or spaces 124 may define a width along the longitudinal axis L, i.e., distance between adjacent protrusions 122, of between 0.25 mm to 3 mm with between 0.3 mm and 2 mm or between 0.3 mm and 1 mm being common, advantageously allowing both collection and retention of the biological sample without the need for an absorbent material, such as cotton fibers.

More specifically, in one embodiment illustrated in FIG. 3, swab body 120 comprises a base 144 in which first end 112 of elongated shaft 110 is disposed. Base 144 has opposing side faces 146 and 147 and opposing end face 148 and 149 that each extend between a first end 150 of base 144 and a second end 151 of base 144. A plurality of the spaced apart protrusions 122 radially outwardly project along each opposing end faces 148 and 149 with a channel or space 124 being bound between each pair of adjacent protrusions 122. In one embodiment, protrusions 122 and channels or spaces 124 are disposed in parallel alignment and project orthogonal to longitudinal axis L. In alternative embodiments, protrusions 122 and channels or spaces 124 can project in other angles. For example, protrusions 122 and channels or spaces 124 can project at an angle in a range between +/−15°, +/−30°, or +/−45° relative to their current orientation. Each protrusion 122 has a top face 152A and an opposing bottom face 152B which in one embodiment can be flat and disposed in parallel alignment. Protrusions 122 also inwardly taper as they project outwardly so as to have an outer curved edge 154. Opposing side faces 146 and 147 of base 144 can be smooth. As such, in one embodiment collecting portion 120 has a transverse cross section normal to longitudinal axis L and passing through protrusions 122 that is elliptical, in the configuration of an elongated circle, or is otherwise elongated having a major axis and a minor axis. In an alternative embodiment, protrusions 122 can also outwardly project from side faces 146 and 147 but have a length shorter than protrusions 122 outwardly projecting from end faces 148 and 149.

As previously discussed, the swab body 120 may be integrally formed with the elongated shaft 110, may be molded to the elongated shaft 110, or may be otherwise attached to the elongated shaft 110 by a friction fit, adhesive, or similar connection. According to the embodiment of FIGS. 1 and 2, the first end 112 of the elongated shaft 110 may have an increased thickness and/or openings for improving a connection with the swab body 120.

Embodiments of the sample collection device 100 may be configured with swab body 120 configured to elastically deform when used for collecting biological samples from a subject. In this respect, swab body 120/protrusions 122 may be formed from a soft, non-absorbent material. Preferably, the collecting portion (swab body) 120 is formed of a material (e.g., elastomer) having a Shore A scale durometer hardness of between 10 A to 70 A, preferably between 15 A to 40 A, and more preferably between 20 A to 30 A. Examples of a soft material of the swab body 120 may include a thermoplastic elastomer (TPE), such as a medical grade thermoplastic polyurethane (TPU), thermoplastic vulcanizate (TPV), or the like. In one embodiment, elongated shaft 110 and swab body 120 can be made from different materials and have different hardness/flexibility properties.

In a currently preferred embodiment, the TPE used to make the swab body is T4MED, which is a specific type of Santoprene® TPE alloy, more particularly a thermoplastic vulcanizate (TPV) consisting essentially of fully cured ethylene propylene diene monomer (EPDM) rubber particles encapsulated in a polypropylene (PP) matrix. T4MED has been found to be particularly well-suited for making the swab body 120 because of its softness, flexibility, and ability to collect saliva and mucus without removing epithelial cells from mucous membrane or other epithelial tissue. Compared to other materials, such as polyurethane foam, viscose fibers, polyether spun fibers, and cotton fibers, it was discovered that making the collecting portion 120 from T4MED reliably collects saliva and mucus and then readily releases the saliva or mucus when the swab body 120 and associated biological sample are contacted with a preservation reagent with little or no mechanical agitation or centrifuging. According to product information published by the manufacturer (Kraiburg TPE GmbH & Co. KG), T4MED has the following materials properties:
Density: 0.880 g/cc;
Shore A hardness: 36.0;
Tensile strength: 8.00 MPa;
Elongation at break: 800%; and
Tear strength: 11.5 kN/m.

Functionally, the particular stiffness of the swab body 120 is advantageously selected to prevent the swab body 120 from stripping epithelial cells from a subject's mucous membranes while collecting a target biological sample in the channels or spaces 124, such as free-floating saliva and/or mucus including proteins and micro-RNA materials. Advantageously, collection of the biological sample in the channels or spaces 124 may be assisted by capillary action, further reducing the need for friction or force to be applied at the collection site of the subject. Accordingly, the swab body 120 of the disclosed embodiments significantly improves collection of a target biological sample by preventing the stripping or collection of epithelial cells from a subject's mucous membranes, greatly enhancing the quality and/or purity of the collected sample and reducing costs and challenges associated with preprocessing or desired analysis of the collected sample.

In certain embodiments, the combined effect of a flexible elongated shaft 110 and a soft, non-absorbent swab body 120 may essentially eliminate the collection of epithelial cells from mucous membranes or other epithelial tissue of a subject, even by unskilled users. This advantage may be achieved while still enabling effective and efficient collection of a biological sample, due to the selection and configuration of materials employed in the sample collection device 100. Notably, the disclosed embodiments also avoid difficulties associated with absorbent materials, from which it may be difficult to extricate sample materials, and materials that may corrupt a sample, such as a cotton or fabric material.

### Multi-Part Sample Collection Systems and Kits Having a Sample Collection Device/Swab

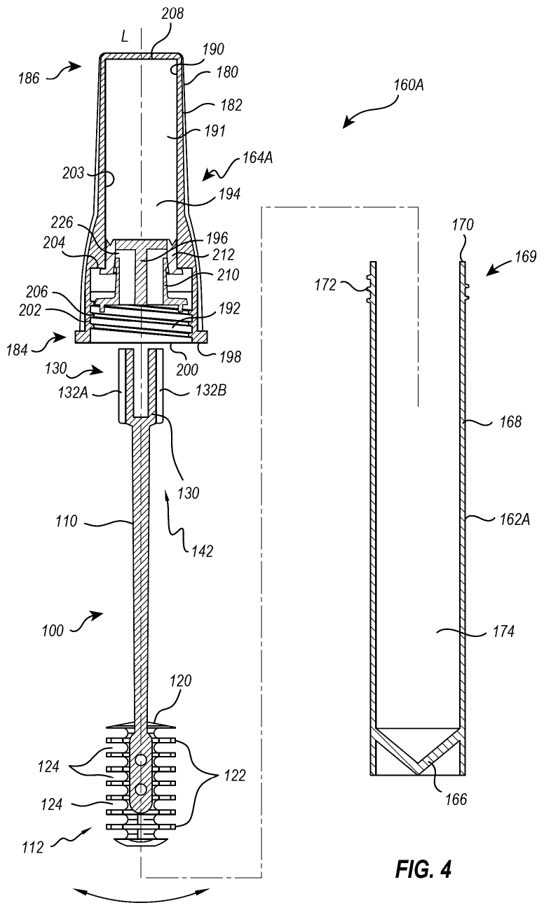

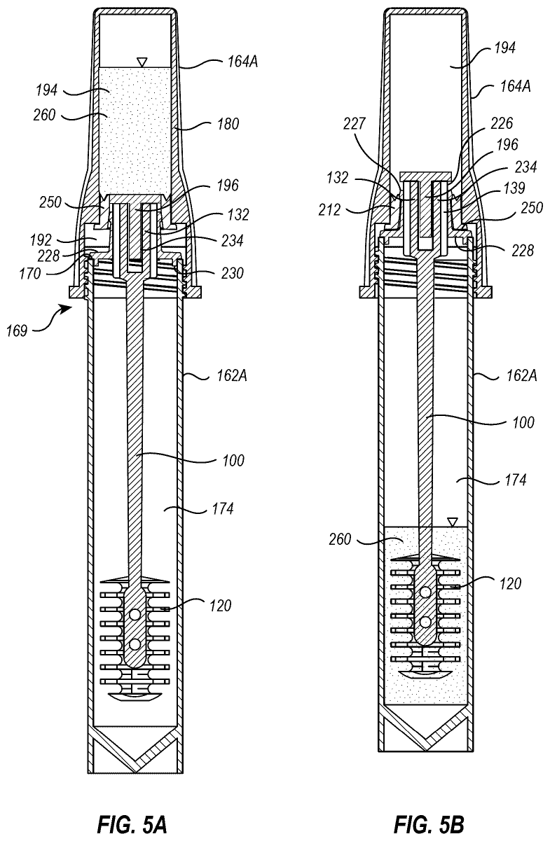

Referring now to FIGS. 4 and 5A-5B, a biological sample may be collected, preserved, and stored in a sample preservation vessel as part of a multi-piece, self-contained sample collection system or kit 160A. A first piece of the system or kit 160A may include a sample preservation vessel 162A, a second piece may include a sealing cap 164A, and a third piece may include sample collection device/swab 100, which may be packaged separately from or removably connected to the sealing cap 164A. The sealing cap 164A may be configured to associate with the sample preservation vessel 162A to seal and preserve the biological sample therein.

According to embodiments, the sealing cap 164A and/or the sample preservation vessel 162A may include a reagent chamber disposed within or integrated therewith. The sealing cap 164A and the sample preservation vessel 162A may be configured such that associating the sealing cap 164A with the sample preservation vessel 162A causes the reagent chamber to be opened, for example by actuating a selectively openable valve or plug. Notably, as described above, the connecting portion 130 of the sample collection device/swab 100 may be configured to associate with the sealing cap 164A without interrupting the valve or plug and without actuating the valve or plug prior to association of the sealing cap 164A with the sample preservation vessel 162A. In particular, the elongated shaft 110 of the sample collection device 100 may be configured with sufficient flexibility to prevent actuation of the valve or plug, as discussed above. In certain embodiments, the connecting portion 130 of the elongate shaft 110 may define holes, grooves or gaps on or between retaining elements 132A and 132B to facilitate passage of fluid from the reagent chamber to the sample preservation vessel 162A.

In some embodiments, the reagent(s) within the reagent chamber may include a preservation or buffering solution that protects the integrity of the probative component of the biological sample prior to purification or testing. Preservation reagents are typically chemical solutions and may contain one or more salts (e.g., NaCl, KCl, Na₂HPO₄, KH₂PO₄, or similar, and which may, in some implementations, be combined as a phosphate buffered saline solution, as known in the art), lysing agents (e.g., detergents such as Triton X-100 or similar), chelating agents (e.g., ethylenediaminetetraacetic acid (EDTA)), ethylene glycol-bis(β-aminoethyl ether)-N,N,N′,N′-tetraacetic acid (EGTA), or similar), distilled water, or other reagents known in the art. Examples of preservation reagents are disclosed in U.S. Pat. Nos. 10,174,362, 10,774,368, and 11,655,495 and U.S. Pat. Pub. Nos. 2021/00711232, 2023/0272368, and 2023/0304071, which are incorporated by reference.

In one or more embodiments, the reagent or buffering solution stabilizes at least one probative component within the biological sample (e.g., nucleic acids, such as DNA and RNA, protein, etc., and combinations thereof) during transfer, transportation, and/or storage at a laboratory, clinic, or other destination. In some embodiments, the biological sample can be stored, at or below room temperature after the preservation solution is added, for weeks or months without significant loss of the probative component. That is, the biological sample can still be utilized for diagnostic, genetic, epidemiologic, or other purposes for which it was collected after storage for weeks or months in the preservation solution.

With continued reference to FIGS. 4 and 5A-5B, in the embodiment depicted, sample preservation vessel 162A comprises a floor 166 and an encircling sidewall 168 upstanding from floor 166. An upper end 169 of sidewall 168 terminates at an annular lip 170. Threads 172 are formed on an exterior surface of sidewall 168 at upper end 169 and are configured to threadedly engage with sealing cap 164A. An interior surface of sidewall 168 encircles and bounds a compartment 174 of sample preservation vessel 162A.

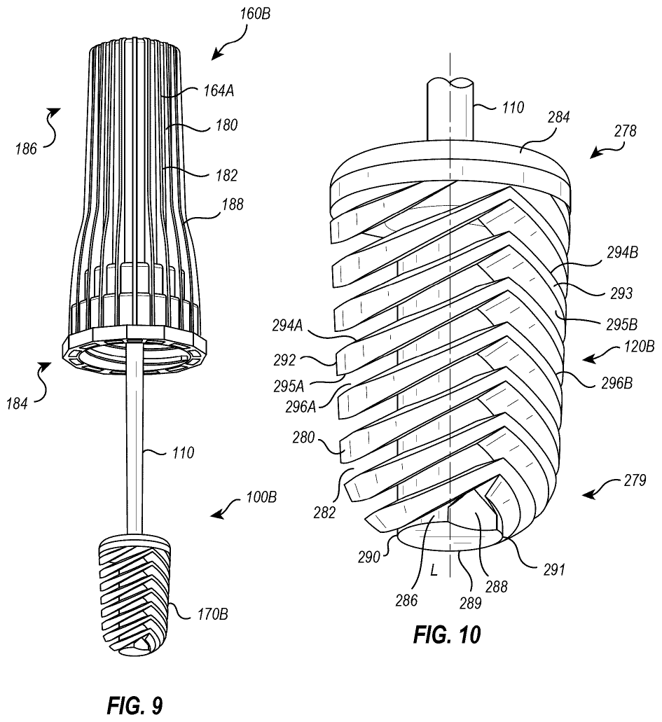

Sealing cap 164A comprises a housing 180 having an exterior surface 182 extending between a first end 184 and an opposing second end 186. In one embodiment, exterior surface 182 has a circular transverse cross section and has linear ribs 188 (FIG. 9) outwardly projecting from exterior surface 182 and longitudinally extending between opposing ends 184 and 186 at radially spaced apart positions around exterior surface 182. Ribs 188 enable firm gripping of sealing cap 164A for twisting onto sample preservation vessel 162A, as discussed below. In alternative embodiment, ribs 188 can be replaced with other forms of surface texture for gripping. Housing 180 also has an interior surface 190 that bounds an interior chamber 191. Sealing cap 164A also optionally includes a valve 196 disposed within interior chamber 191. Valve 106 divides interior chamber 191 into a receiving chamber 192 and a reagent chamber 194. Valve 106 is selectively movable between a closed position and an open position to selectively control fluid communication between reagent chamber 194 and receiving chamber 192.

More specifically, first end 184 of housing 180 terminates at an annular lip 198 that encircles and opening 200 to receiving chamber 192. Interior surface 190 includes a first interior surface portion 202 that extends from annular lip 198 to a radially inwardly projecting annular shoulder 204. First interior surface portion 202 encircles at least a portion of receiving chamber 192 and has threads 206 formed thereon that are configured to threadedly engage with threads 172 on sample preservation vessel 162A. Interior surface 190 also includes second interior surface portion 203 that extends from shoulder 204 to a terminal end wall 208 at second end 186 of housing 180. Second interior surface portion 203 encircles at least a portion of reagent chamber 194.

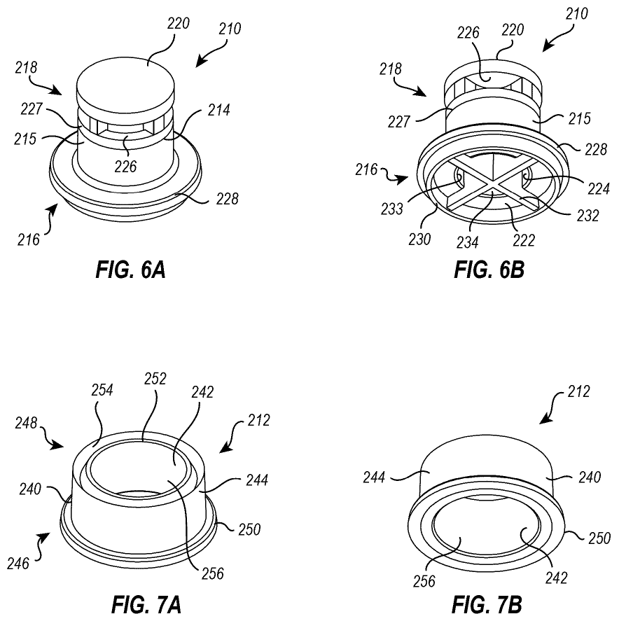

Valve 196 comprises a core 210 and an annular collar or sleeve 212 that encircles core 210. As depicted in FIGS. 6A and 6B, core 210 comprises a stem 214 having an exterior surface 215 extending from a first end 216 to an opposing second end 218. Second end 218 terminates at an end wall 220. First end 216 terminates at an end face 222. A cavity 224 is recessed into end face 222 and extends to end wall 220. A flange 228 encircles and radially outwardly projects from exterior surface 215 at first end 216. An annular guide lip 230 projects downwardly from a bottom surface of flange 228 so as to encircle cavity 224. An X-shaped brace 232 is disposed within cavity 224. Brace 232 extends along an interior surface 233 of stem 214 to end wall 220. Brace 232 divides cavity 224 into four separate channels 234A-D (see FIG. 8B) that each extend along the length of cavity 224. An annular vent opening 226 passes through stem 214 below end wall 220 and communicates with each channel 234A-D at second end 218. As such, fluid can flow into vent opening 226 at second end 218, along each of channels 234A-D and then exit core at first end 216. Encircling and outwardly projecting from the exterior surface of stem 214 directly below vent opening 226 is an annular seal ring 227. As discussed below, seal ring 227 can help to produce a liquid tight seal between core 210 and collar 212.

Turning to FIGS. 7A and 7B, collar 212 comprises a tubular sleeve 240 having an interior surface 242 and an opposing exterior surface 244 extending between a first end 246 and an opposing second end 248. A retaining ring 250 encircles and radially outwardly projects from first end 246. Interior surface 242 bounds a passage 256 passing through sleeve 240 between first end 246 and second end 248. As discussed below, passage 256 is configured to receive stem 214 of core 210. Second end 248 terminates at an end face 252. An annular groove 254 is recessed into end face 252 so as to encircle passage 256.

With reference to FIGS. 4, 6A, 6B, 7A, and 7B, valve 196 is assembled by pressing second end 218 of stem 214 into passage 256 of collar 212/sleeve 240 at first end 246. Stem 214 is advanced until end wall 220 of core 210 is disposed at or adjacent to second end 248 of collar 212/sleeve 240. In this position, vent opening 226 is covered and sealed closed by interior surface 242 of collar 212/sleeve 240. Core 210 and collar 212 are sufficiently tightly friction fitted together in this position so as to prevent fluid from leaking therebetween. This sealed engagement can be produced by seal ring 227 of core 210 biasing in sealed engagement against the interior surface of collar 212/sleeve 240.

In one method of assembly, valve 196 is coupled to housing 180 of sealing cap 164A by advancing assembled valve 196 into receiving chamber 192 and toward reagent chamber 194. Specifically, collar 212/sleeve 240 is advanced toward reagent chamber 194 so that exterior surface 244 of collar 212/sleeve 240 is friction fit in sealing engagement against second interior surface portion 203 of housing 180. Collar 212/sleeve 240 is advanced concurrently with core 210 until retaining ring 250 of collar 212 is stopped against shoulder 204 of sealing cap 164A. Valve 196 is then securely disposed within sealing cap 164A in a closed position. In an alternative method of assembly, collar 212 can first be secured within housing 180, as discussed above, and then core 210 received therein so that vent opening 226 is covered and sealed closed by collar 212/sleeve 240. In either method of assembly, collar 212 can be snap-fittingly received into the housing 180 of the sealing cap 164A, creating a fluid tight connection therebetween. Retaining ring 250 of collar 212 engages with housing 180 of sealing cap 164A to stabilize the collar 212 and can assist with the snap-fit connection. In an alternative embodiment, collar 212 or the corresponding shape of collar 221 can be integrally formed within housing 180 for engagement with core 210, thereby avoiding the need for attachment of separate collar 212.

The portion of interior chamber 191 disposed between opening 200 and valve 196 comprises the receiving chamber 192 and the portion of interior chamber 191 disposed between valve 196 and end wall 208 comprises the reagent chamber 194. The preservation reagent is disposed within reagent chamber 194 prior to securing valve 196 in place. When valve 196 is in the closed position, as shown in FIGS. 4 and 5A, fluid communication between reagent chamber 194 and receiving chamber 192 is sealed closed. Controlled opening of valve 196 will be discussed below with reference to FIGS. 5A-5B.

As previously discussed, in one embodiment sample collection device 100 can be coupled to sealing cap 164A or some other form of handle. For example, the connecting portion 130 may include one or more retaining elements 132 shaped to correspond with recesses or openings defined by a base portion of the sealing cap or handle. In use, the retaining elements 132 may be inserted into the recesses or openings in order to form a friction fit between the connecting portion 130 and the sealing cap or handle.

In varying embodiments, the retaining elements and/or the base portion may include friction enhancing shapes, protrusions, materials, or the like, such as to improve the friction fit. For example, a retaining element may be provided in the form of an annular or cone shape, such as in the form of a funnel having a closed end at the second end of the elongated body or a ring connected to the second end of the elongated body. In another example, the connecting portion may comprise two or more retaining elements projecting from the second end of the elongated body in a generally Y- or cone-shaped profile, such that the retaining elements may be elastically deformed (e.g., expanded or compressed relative to the longitudinal axis) to fit within or be fixed to sealing caps or handles of variable dimensions and shapes.

In preferred embodiments, the retaining elements 132 may be configured for use with a plurality of existing sealing caps and corresponding sample collection and preservation vessels. Additional sealing caps are contemplated, such as disclosed in U.S. Pat. Pub. No. 2020/0254460 and U.S. Pat. Nos. 11,712,692, 11,701,094, 10,973,497, 10,619,187, 9,523,115, 8,728,414, and 7,482,116, which are herein incorporated by reference in their entirety.

In the depicted embodiment, sample collection device 100 is specifically coupled to valve 196 which forms a portion of sealing cap 164A. For example, depicted in FIG. 8B is a bottom view of valve 196/core 210 showing brace 232 dividing cavity 224 into channels 234A-D. Brace 232 is comprised of two panels 238A and 238B that centrally intersect so as to be orthogonal to each other. Brace 232 includes four radially spaced apart corner faces 236A-D with each corner face 236A-D at least partially bounding a corresponding channel 234A-D, respectively. Each corner face 236A-D has a 90° angle and is shaped complementary to inside face 136 of retaining elements 132A and B of sample collection device 100/body 110 (see FIG. 8A). During assembly, as depicted in FIG. 8C, the free ends of retaining elements 132A and 132B are slid into opposing channels 234A and 234C (or alternatively opposing channels 234B and 234D). Inside face 136 of retaining elements 132A and 132B sit flush against complementary corner faces 236A and 236C so as to produce a secure but releasable friction engagement therebetween. In one embodiment, retaining elements 132A and 132B can be configured to pinch brace 232 therebetween.

The L-shaped configuration of retaining elements 132A and B achieves a number of benefits. For example, the L-shape maximizes surface engagement between retaining elements 132A and B and corner faces 236 while minimizing the occlusion of channels 234. That is, even with retaining elements 132A and 132B disposed within channels 234A and 234C, fluid can still flow through channels 234A and 234C by passing along grooves 139 of retaining elements 132A and 132B. Brace 232 is also uniquely configured in that it optimizes the reinforcing of stem 214 which is radially inwardly compressed by collar 212 (see FIG. 4) while still maximizing the area of channels 234A-D for optimal fluid flow.

In alternative embodiments, however, it is appreciated that retaining elements 132 and brace 232 can have a variety of different configurations. For example, in alternative embodiments retaining elements 132 can have alternative cross-sectional configurations such as square, rectangular, triangular, pie shaped, circular, or other polygonal or irregular shapes. Furthermore, in contrast to having just two retaining elements 132, elongated body/shaft 110 can also be formed with three or four forked retaining elements 132 that are configured to slide into a corresponding one of channels 234. In still other embodiments, brace 232 can be formed by only a single panel 238 or from three, four or more panels 238 that all intersect at a central point to form a pie shaped configuration. In any of these embodiments, retaining elements 132 can be shaped to fit within the correspondingly formed channels 234.

Enabling elongated shaft 110/sample collection device 100 to be removably attached to valve 196/sealing cap 164A has a number of benefits. For example, forming sample collection device 100 and valve 196/sealing cap 164A as two separate elements makes it easier to produce both elements which are commonly made from different materials. Furthermore, making sample collection device 100 removable from valve 196/sealing cap 164A enables collection device 100 to be used independently of valve 196/sealing cap 164A, both during the process of collecting the biological sample and/or in the subsequent storage or processing of the biological sample, which can be beneficial in some situations. However, in other embodiments, depending on the application, it can be desirable for elongated shaft 110/sample collection device 100 to be permanently attached or formed with valve 196/sealing cap 164A. This can be accomplished by using adhesive, welding, press fit connection, overmolding or other techniques to secure elongated body 110/sample collection device 100 to valve 196/sealing cap 164A. In still other embodiments, elongated shaft 110/sample collection device 100 can be integrally molded as a single unitary member with valve 196/sealing cap 164A.

FIGS. 5A and 5B illustrate the operation of valve 196. Specifically, in FIG. 5A, valve 196 is secured within housing 180 and is in the closed position (as shown in FIG. 4). Sample collection device/swab 100 is also secured to valve 196/sealing cap 164A by having retaining elements 132 disposed within channels 234. A preservation reagent 260, such as one of those previously discussed above, is sealed within reagent chamber 194. The fluid tight connection between the collar 212 and the core 210 and between the housing 180 and collar 212 prevents the premature or unintentional expulsion of reagent 260 from the reagent chamber 194.

Upper end 169 of vessel 162A is partially threaded into receiving chamber 192 so that lip 170 is disposed against flange 228 of valve 196. Guide lip 230 helps with proper positioning and centering of lip 170 on valve 196. In this position, sample collection device 100, which would typically already have a biological sample collected on collecting portion (swab body) 120, is disposed within compartment 174 of vessel 162.

Next, to move valve 196 to the open position, vessel 162A is manually threaded further up into receiving chamber 192 by rotating sealing cap 164A and vessel 162A relative to each other. In so doing, lip 170 of vessel 162A pushes against flange 228 causing core 210 (with sample collection device 100 attached thereto) to slide upward within and relative to collar 212. Core 210 is advanced to place vent opening 226 into fluid communication with reagent chamber 194. In one embodiment, core 210 can be advanced until flange 228 bases against retaining ring 250. In this open position with vent opening 226 communicating with reagent chamber 194, reagent 260 can freely flow through vent opening 226, down through channels 234A-D (and/or along grooves 139 of retaining elements 132 where retaining elements 132 are disposed within channels 234) and into compartment 174 of vessel 162A.

As the valve 196 transitions from the closed position to the open position, the annular seal ring 227 disposed on the core 210 forms a fluid tight seal with the interior surface 242 of the collar 212. Upon fully entering the open position, the annular seal ring 227 is flush with or adjacent to end face 252 of the collar 212 and maintains a fluid-tight connection therebetween. Accordingly, there is no or minimal pooling of reagent 260 between interior surface 242 of the collar 212 and the exterior sidewall of the core 210. Instead, all or substantially all of reagent 260 is directed from the reagent chamber 194, through the vent opening 226 and channels 234A-D, and into vessel 162A. Further disclosure with regard to valve 196, the operation thereof, and alternatives thereof, which can be used in the present invention, are disclosed in U.S. Pat. No. 11,701,094, which is incorporated by reference.

As reagent flows into compartment 174, it can flow over collecting portion 120 to help separate the biological sample from collecting portion 120. The combined vessel 162A and sealing cap 164A can also be shaken, rocked, or otherwise manipulated to help separate the biological sample from collecting portion 120 and into reagent 260. It should be appreciated that in some embodiments, the vent opening 226, channels 234A-D and/or grooves 139 and/or other structure of the core 210 can beneficially act as an agitator of fluids entering and/or traversing between reagent chamber 194 and compartment 174 of sample preservation vessel 162A.

It is understood that sealing cap 164A and sample preservation vessel 162A are only one example sealing caps and sample collection vessels that can be used with sample collection device 100. In alternative embodiments, it is appreciated that a variety of different configurations of sealing caps and/or sample preservation vessels can be used with sample collection device 100 or alternatives of sample collection devices disclosed herein. For example, the sealing caps and/or sample collection and preservation vessels disclosed in U.S. Pat. Pub. Nos. 2020/0254460, 2021/0085296, 2020/0397418, and 2020/0156056 and U.S. Pat. Nos. 11,712,692, 11,701,094, 10,973,497, 10,619,187, 9,523,115, 8,728,414, and 7,482,116, which are incorporated by reference, can be used with sample collection device 100 and/or other alternative sample collection devices disclosed herein. The skilled person can, in view of the present disclosure, make appropriate modifications to the sample collection device, sealing cap, and/or sample collection and preservation vessel as needed to yield an operational device.

FIGS. 9 and 10 illustrate an alternative embodiment of a self-contained sample collection system or kit 160B. Like elements between sample collection system or kit 160B and sample collection system or kit 160 are identified by like reference characters. Sample collection system or kit 160B includes sealing cap 164A, as previously discussed, sample preservation vessel 162A (see FIG. 4) which associates with sealing cap 164A, as previously discussed, and a sample collection device 100B coupled with and projecting from sealing cap 164A. Sample collection device 100B comprises elongated body/shaft 110 which can be removably or securely fixed to sealing cap 164A in the same alternative manners as previously discussed with regard to sample collection system or kit 160A. Mounted on the end of elongated shaft 110 is a collecting portion (swab body) 120B.

Swab body 120B has an alternative configuration relative to swab body 120 but can be made of the same non-absorbing, soft materials, as previously discussed. In general, as shown, swab body 120B extends between a first end 278, from which elongated shaft 110 projects, and an opposing second end 279. Swab body 120B includes a plurality of protrusions 280 and a plurality of channels or spaces 282, each channel or space 282 being bound between an adjacent pair of protrusions 280. In varying embodiments, the protrusions 280 may be configured to extend outward from the elongated shaft 110 at an angle of less than 90° from central longitudinal axis L a direction toward second end 279, more particularly at an angle of 80° or less, an angle of 60° or less, or preferably at an angle of about 45° or less. In this manner, the channels or spaces 282 may be advantageously oriented for both collection and release of a biological sample. For example, during insertion to a subject's nose, mouth or throat, in addition to a capillary effect, the channels or spaces 282 can advantageously “scoop” from a collection site. In addition, the channels or spaces 282 advantageously facilitate release of the collected sample when a reagent or solution is applied thereto, particularly when the sample collection device 100B may be oriented in a downward direction. In this manner, a biological sample may be more easily separated from the sample collection device without a reduced or eliminated need for centrifugation, dramatically reducing the costs of collecting and analyzing biological samples, while enabling collection and analysis to be performed in areas with limited infrastructure, such as without centrifuges and trained technicians.

In some embodiments, protrusions 280 may be configured as opposing ridges, as seen in FIG. 10. For example, pairs of opposing ridges may be configured to meet at an angle of less than 90° in a direction of the second end 279, more particularly at an angle of 80° or less, at an angle of 60° or less, preferably at an angle of about 45°. This arrangement may provide that the channels or spaces 282 formed by the ridges or protrusions 280 are better exposed, for example at two of four lateral sides, for collecting and releasing the biological sample than may result from channels having a shape of a surface of a cone where an interior portion is essentially closed in, such as may be the case with annular ridges or the like.

Embodiments of the swab body 120B may include a proximal flange 284 having an increased thickness relative to the protrusions 280. The increased thickness of the proximal flange 284 may advantageously prevent the swab body 120B from entirely losing its shape in response to pressure, such as may occur during insertion or removal of the sample collection device from a collection site.

With continued reference to FIGS. 9 and 10, swab body 120B can be described in more detail as comprising a base 286 having opposing side faces 288 and 289 and opposing end face 290 and 291 that each extend between first end 278 and second end 279. Longitudinal axis L extends along the length of base 286 and elongated body 110 is secured to base 286 in any of the same manners as previously discussed with regard to sample collection device 100 so as to outwardly project form first end 278. Base 286 typically has a transverse cross section that is circular, elongated circular, or elliptical. However, other configurations can also be used.

The plurality of spaced apart protrusions 280 encircle and outwardly project from base 286 so that they are spaced apart along axis L. Channel or space 282 is formed between each adjacent pair of protrusions 280. Protrusions 280 can be spaced apart by a distance in a range between of between 0.25 to 3 mm with between 0.3 and 2 mm or between 0.3 and 1 mm being common, advantageously allowing both collection and retention of the biological sample using capillary action without the need for an absorbent material. Other spacing can also be used. Swab body 120B can be formed with between 3 and 15 spaced apart protrusions 280 with between 3 and 10 or between 3 and 8 being more common. Other numbers can also be used. Protrusions 280 are configured so that when swab body 120B is viewed in plan view from second end 279, swab body 120B has an outer perimeter that is elliptical or in the configuration of an elongated circle. Swab body 120B can also inwardly taper at second end 279. Proximal flange 284 encircles and radially outwardly projects from first end 278 of base 286.

Each protrusion 280 can comprise a first ridge 292 that outwardly projects from side face 288, end face 290 and side face 289 and a second ridge 293 that outwardly projects form side face 288, end face 291 and side face 289. The first ends of first ridge 292 and second ridge 293 join together at side face 288 while second ends of first ridge 292 and second ridge 293 join together at side face 289. First ridge 292 has a top face 294A and an opposing bottom face 295A that can be disposed in parallel alignment. First ridge 292 with top face 294A and bottom face 295A slope at a downward angle toward second end 279 as they extend from side face 288 and 289 toward end face 290. First ridge 292 can be sloped so that an inside angle is formed between axis L and first ridge 292, top face 294, and/or bottom face 295 that is 80° or less, 60° or less, or preferably 45° or less.

Second ridge 293 is similarly configured to first ridge 292 but slopes in the opposite direction. That is, second ridge 293 has a top face 294B and an opposing bottom face 295B that can be disposed in parallel alignment. Second ridge 293 with top face 294B and bottom face 295B slope at a downward angle toward second end 279 as they extend from side face 288 and 289 toward end face 291. Second ridge 293 can be sloped so that an inside angle is formed between axis L and second ridge 293, top face 294B, and/or bottom face 295B that is 80° or less, 60° or less, or preferably 45° or less.

A channel section (or space) 296A is disposed between each pair of adjacent first ridges 292 and a channel section 296B is disposed between each pair of adjacent second ridges 293. Channel sections (or spaces) 296A and channel sections (or spaces) 296B can be disposed at the same angular orientation as first ridges 292 and second ridges 293, respectively, as discussed above.

First ridges 292 and second ridges 293 meet together at side face 288 and side face 289 so that an inside angle is formed therebetween that is 90° or less, more particularly at an angle of 80° or less, at an angle of 60° or less, or preferably at an angle of 45° or less.

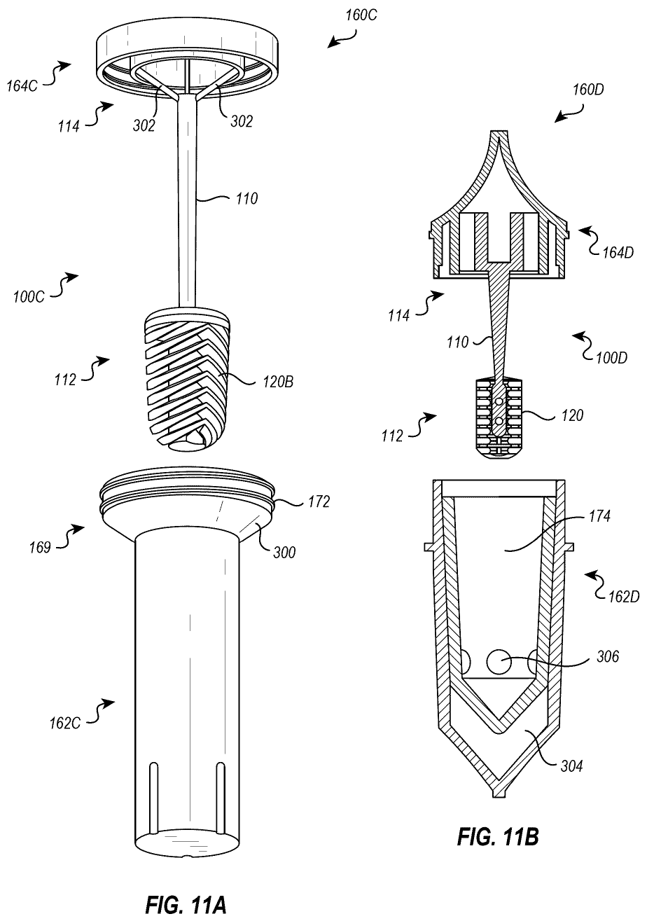

FIGS. 11A and 11B illustrate two alternative embodiments of a self-contained sample collection systems or kits 160C and 160D, respectively. Like elements between the prior discussed sample collection systems or kits and sample collection systems or kits 160C and 160D are identified by like reference characters.

Sample collection systems or kit 160C includes a sealing cap 164C, a sample preservation vessel 162C which associates with sealing cap 164C, and a sample collection device 100C projecting from sealing cap 164C. Sample preservation vessel 162C differs from sample preservation vessel 162A in that it has a radially enlarged mouth 300 at upper end 169 having threads 172 formed thereon. Enlarged mouth 300 can function as a funnel or guide for assistance in delivering a reagent, biological specimen, collecting portion, or any other element therein. It is appreciated that the sample preservation vessel 162C can have a variety of different configurations and sizes.

Sealing cap 164C is configured to threadedly join with sample preservation vessel 162C to form a sealed engagement therebetween. However, in contrast to sealing cap 164A, sealing cap 164C does not include a valve or a reagent chamber. Sample collection device 100C includes an elongated body/shaft 110 having collecting portion/swab body 120B coupled thereto at first end 112. Disposed at a second end 114 of elongated shaft 110 are retaining elements 302, which permanently secure elongated shaft 110 to sealing cap 164C.

Sample collection system or kit 160D includes a sealing cap 164D, a sample preservation vessel 162D which associates with sealing cap 164D, and a sample collection device 100D projecting from sealing cap 164D. Sample preservation vessel 162D differs from sample preservation vessel 162A in that it includes both compartment 174 for receiving a collecting portion and also a reagent chamber 304 disposed below compartment 174. Reagent chamber 304 communicates with compartment 174 through ports 306. Sealing cap 164D does not include a reagent chamber or valve but is configured to threadedly join with sample preservation vessel 162D to form a sealed engagement therewith. Sample collection device 100D includes elongated body/shaft 110 having collecting portion/swab body 120 formed at first end 112 for receiving in compartment 174 and having second end 114 coupled to sealing cap 164D. In view of the foregoing, it is appreciated that the present invention envisions that a variety of different configurations of sample collection systems or kits can be formed by mixing and matching all available variations of sample collection and preservation vessels, sealing caps, and sample collection devices.

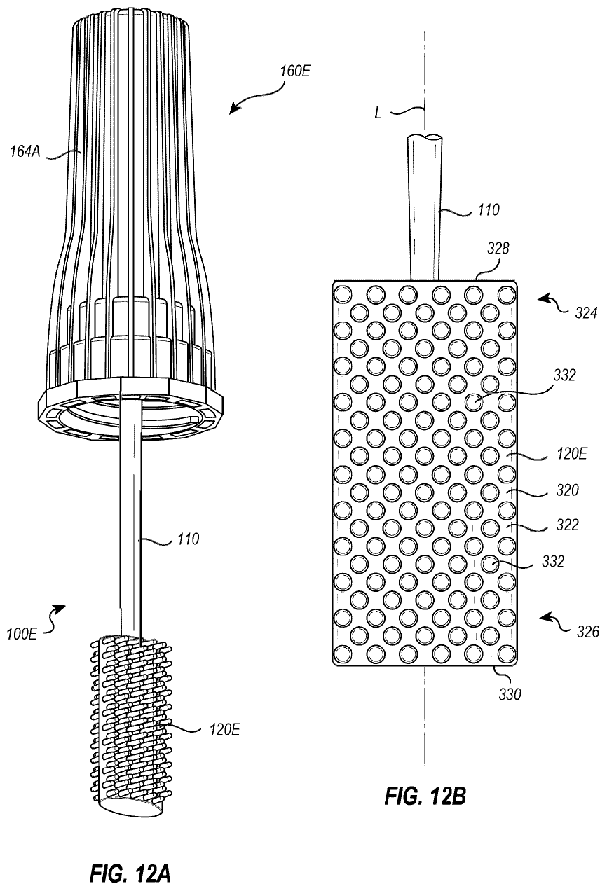

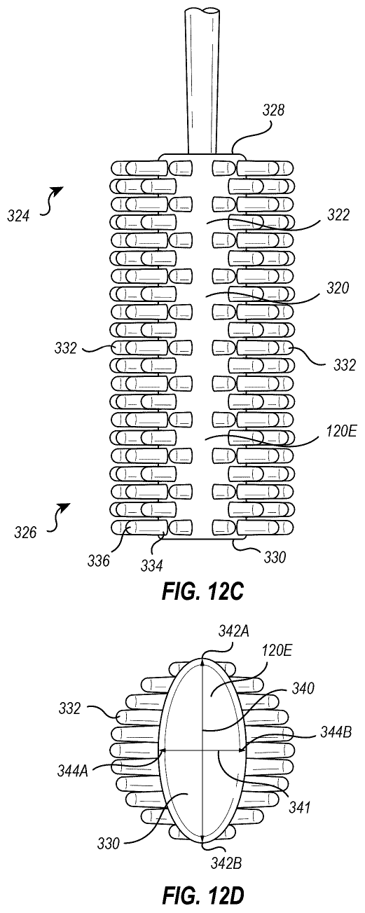

Depicted in FIG. 12A is an alternative embodiment of a self-contained sample collection system or kit 160E. Like elements between the prior discussed sample collection systems or kits and sample collection system or kit 160E are identified by like reference characters. Sample collection system or kit 160E includes sealing cap 164A, as previously discussed, sample preservation vessel 162A (as shown in FIG. 4) which associates with sealing cap 164A, as previously discussed, and a sample collection device 100E coupled with and projecting from sealing cap 164A. Sample collection device 100E comprises elongated body/shaft 110 which can be removably or securely fixed to sealing cap 164A in the same alternative manners as previously discussed with regard to sample collection system or kit 160A. Mounted on the end of elongated body/shaft 110 is a collecting portion/swab body 120E.

Swab body 120E has an alternative configuration relative to the swab bodies previously discussed herein. As depicted in FIG. 12A-12D, swab body 120E comprises a base 320 having an encircling perimeter face 322 extending between a first end 324 and an opposing second end 326. First end 324 terminates at a proximal end face 328 while second end 326 terminates at a distal end face 330. First end 112 of elongated shaft 110 (FIG. 2) is disposed within base 320/swab body 120E, such as in one of the manners as previously discussed with regard to sample collection device 100, so that elongated shaft 110 outwardly projects from proximal end face 328. Longitudinal axis L passing along elongated shaft 110 centrally passes through base 320 between proximal end face 328 and distal end face 330. In one embodiment, one or both of proximal end face 328 and distal end face 330 can be planar and disposed orthogonal to longitudinal axis L. Base 320 typically has a length extending between proximal end face 328 and distal end face 330 that is at least or less than 0.5, 1, 1.5, 2, 2.5, 3, 4 cm or is in a range between any two of the foregoing values. Other dimensions can also be used. The length of base 320 can, in part, depend on the intended application of swab body 120E.

In the depicted embodiment, base 320 has a transverse cross section normal to longitudinal axis L having an elliptical configuration, an elongated circular configuration, or some other elongated configuration having a major axis and a minor axis. The transverse cross-sectional configuration can be constant over the length between proximal end face 328 and distal end face 330 or constant over at least a majority of the length between proximal end face 328 and distal end face 330. As such, a bottom end view of base 320 can also have the same configuration. As perhaps best depicted in FIG. 12D, the transverse cross section has a major axis 340 extending between opposing end faces 342A and 342B with a maximum diameter and a minor axis 341 extending between opposing side faces 344A and 344B with a minimum diameter. In one embodiment, the maximum diameter is at least or less than 4, 6, 8, 10, 12, 14, or 16 mm or is in a range between any two of the foregoing values and the minimum diameter is at least or less than 2, 3, 4, 6, or 8 mm or is in a range between any two of the foregoing values. Other dimensions can also be used depending upon the application. In one embodiment, the maximum diameter is at least or less than 1.25, 1.5, 1.75, 2, 2.5, 3, 3.5, or 4 times greater than the minimum diameter.

As again shown in FIGS. 12A-12D, outwardly projecting from encircling perimeter face 322 are a plurality of elongated protrusions 332. Each protrusion 332 has a first end 334 disposed on base 320 and a freely disposed second end 336. Second end 336 can be rounded such as having a semispherical configuration. Each protrusion 332 has a transverse cross section that is circular. However, in alternative embodiment, the transverse cross section could be elliptical, oval, polygonal or a compound configuration having curved and flat faces. Protrusions 332 typically have a circular cross section that inwardly tapers as it extends from first end 334 to second end 336, e.g., can have a frustoconical configuration. In other embodiments, protrusions 332 can have a cylindrical configuration with rounded second ends 336.

In the depicted embodiment, protrusions 332 outwardly project from perimeter face 322 so as to extend perpendicular to major axis 340, i.e., they extend perpendicular to a plane extending through major axis 340 parallel to longitudinal axis L of elongated body 110. The length of protrusions 332 can vary. For example, protrusions 332 can be longer toward the center of side faces 344A and 344B and progressively or periodically shorter toward end faces 342A and 342B. In other embodiments all or a majority of protrusions 332 can be the same length. For example, in one embodiment, only protrusions 332 directly adjacent to end faces 342A and 342B are shorter while the remainder are the same length. As a result, in at least some embodiments, a bottom view or transverse cross-sectional view of swab body 120E, normal to axis L, based on the terminal ends of protrusions 332, can have a circular configuration or a configuration that is at least more circular than the transverse cross section of base 320. In one embodiment, protrusions 332 can have a length in a range between 1 mm and 8 mm with between 2 mm and 6 mm being more common. Protrusions 332 can also have an average diameter or width in a range between 0.2 mm and 1.2 mm with between 0.3 mm and 1 mm and between 0.3 and 0.8 being more common. Protrusions 332 are also typically spaced apart by a distance between 0.3 mm and 2 mm with between 0.3 mm and 1.5 mm or between 0.5 mm and 1 mm being more common. Other dimensions for each of the foregoing can also be used depending on the application. Each opposing side of base 320 will commonly have at least 30, 40, 50, 60, or 80 protrusions 332 formed thereon. Other numbers can also be used depending on the application. Swab body 120E can be made of the same non-absorbing, soft materials, as previously discussed with regard to collecting portion 120.

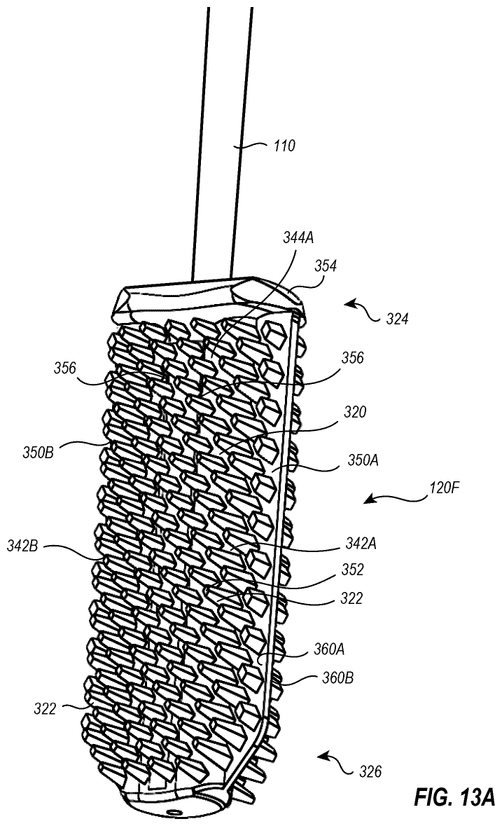

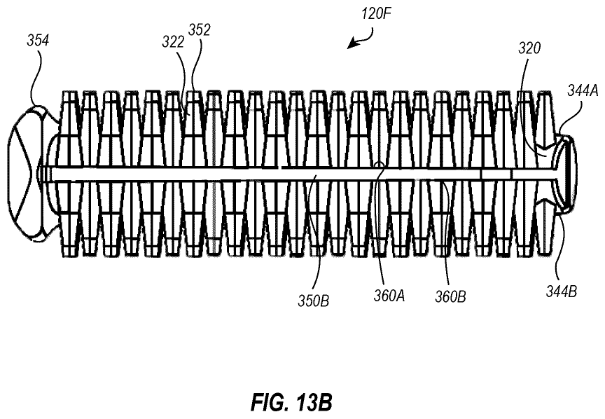

Depicted in FIGS. 13A and 13B is a collecting portion/swab body 120F that has been modified relative to collecting portion/swab body 120E. Like elements between collecting portions/swab bodies 120F and 120E and are identified by like reference characters. Unless otherwise identified, all of the disclosure, dimensions, numbers, alternatives, materials, and the like discussed with swab body 120E are also applicable to swab body 120F.

Swab body 120F includes base 320 as discussed above. However, in this embodiment, optional flanges 350A and 350B outwardly project from end faces 342A and 342B, respectively, and linearly extend from first end 324 to second end 326. Each flange 350A and 350B has a first side face 360A and an opposing second side face 360B. Side faces 360A and 360B can be planar and disposed in parallel alignment. If desired, one, two or more spaced apart, optional grooves 356 can be formed on side face 344A and/or 344B that linearly extend from first end 324 to second end 326. For example, some of the grooves 356 can add flexibility and/or promote bending along the grooves 356. Swab body 120F also includes protrusions 332 outwardly projecting from opposing sides of base 320 and, more specifically, outwardly projecting from opposing sides of base 320 so as to project normal to major axis 340 (FIG. 12D), i.e., they extend perpendicular to a plane extending through major axis 340 parallel to longitudinal axis L of elongated body 110. In the depicted embodiment, protrusions 332 also outwardly projecting from opposing sides of each flange 350A and 350B. Protrusions 332 on flange 350A and 350B can extend parallel to protrusions 332 extending from base 320. However, in contrast to swab body 120E, in which protrusions 332 are longer at the center of side faces 344A and 344B and get shorter toward end faces 342A and 342B (FIG. 12D), in swab body 120F, protrusions 332 are shorter at the center of side faces 344A and 344B and get longer toward end faces 342A and 342B. Specifically, in one embodiment, protrusions 332 can be sized so that a majority of the free terminal ends of protrusions 332 are disposed in common planes on opposing sides of swab body 120F. For example, in one embodiment, the free terminal ends of all protrusions 332 on a side of swab body 120F can be disposed in a common plane except for only an outer row of protrusions 332 on each flange 350A and 350B which can be shorter than the adjacent protrusions 332.

Swab body 120F also differs from swab body 120E in that protrusions 332 on swab body 120F have a polygonal transverse cross section and terminate at flat end faces 352. For example, protrusions 332 on swab body 120 can have a transverse cross section the is square, rectangular, a rhombus, a diamond, or a parallelogram. Protrusions 332 could also be formed having a polygonal transverse cross section having other numbers of sides such as 3, 5, 6, 8, or more. Protrusions 332 can be formed with a polygonal transverse cross section to help increase surface area which can further help in retention of the biological sample during the collection process. Finally, swab body 120F can be formed with an annular proximal ridge 354 disposed proximal of base 320 and projecting radially outward past base 320.

The foregoing designs of the collecting portions/swab bodies, and particularly collecting portions/swab bodies 120E and 120F, have been found to produce a number of unique advantages. For example, the configuration and spacing of the protrusions has been found to significantly improve collection of a biological sample, such as saliva, and release of the biological sample into a reagent. The collection and retention of the biological sample is achieved through configuring and spacing of the protrusions so as to optimize surface area and capillary action used in collection and retention but without producing an undue retention force. For example, conventional foam swabs, cotton swabs, polyester spun fiber swabs, and other swabs known in the art for collecting saliva require that the swab be subjected to centrifugation to achieve adequate release of the biological sample. However, the collecting portions/swab bodies disclosed herein, and particularly those disclosed in FIGS. 12 and 13, can freely release the biological sample without the required use of centrifugation. For example, the biological sample can be released by simply flowing the reagent over the collecting portion/swab body and/or by manually shaking or otherwise manipulating a vessel, such as vessel 162 (FIG. 4), having the collection portion/swab body therein, so that the reagent freely washes over the collection portion/swab body to release the biological sample.

In addition, the configuration of base produces collection portions with opposing broad surfaces for collecting the biological specimen but a relatively narrow profile for easy insertion and manipulation. The collection portions/swab bodies in all embodiments can also be made of the same soft, flexible, non-absorbing materials as previously discussed with regard to swab body 120, thereby limiting scraping or the removal of cells while collecting the biological samples.

### EXAMPLES

### Example 1

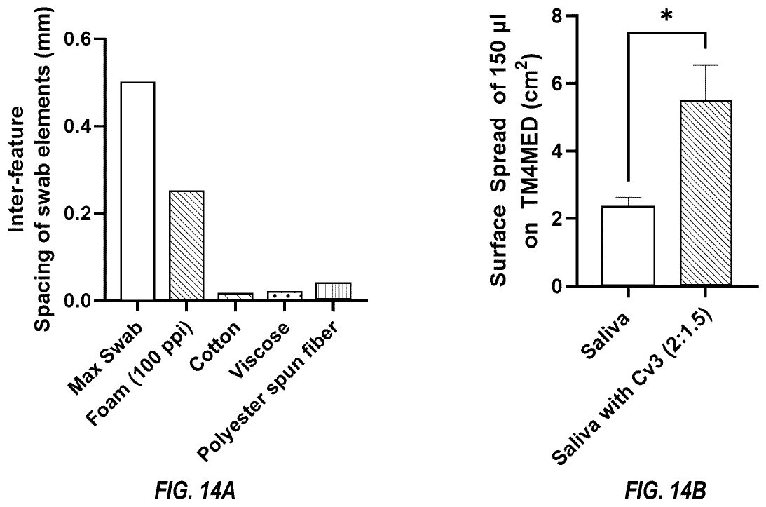

Five sample collection swabs were considered and their properties compared with respect to variables that affect how well they are able to initially collect and retain a biological sample and then release the sample upon contacting the swab with a preservation reagent. FIG. 14A graphically illustrates structural differences of swab elements of a preferred sample collection swab embodiment within the scope of the disclosure and other swabs. FIG. 14B graphically illustrates how the surface tension of the preferred swab element changes when contacted with either saliva by itself or saliva and preservation reagent.

FIG. 14A more particularly is a chart showing the inter feature spacing of various swabs bodies, including Max Swab, which is an example of an inventive swab body made from thermoplastic elastomer (i.e., T4MED), and other common swab materials, including polymer foam (used in products sold by Oasis Diagnostics and DNA Genotech), cotton flocked swab, viscose, and polyester spun fiber. The spacing between the swab collection elements (e.g., projections, pores, fibers, etc.) are substantially greater in the MaxSwab compared to the polymer foam swab and even more substantially greater compared to the cotton, viscose, and polyester spun fiber swabs. As will be discussed below, spacing between swab collection elements coupled with swab material significantly affect the ability of the swab device to collect, retain, and release a biological sample when bathed in preservation reagent.

FIG. 14B graphically illustrates the results of a surface spread test, which demonstrates that dilution of saliva with a preservative reagent had a profound impact on surface tension and subsequently on sample-swab interactions. Using a sheet of T4MED material (Santoprene® TPE alloy used to make Max Swab), 150 μl droplets of saliva and saliva mixed with a commercial preservation reagent (Cv3) used by Spectrum Solutions in sample collection devices were placed at a 2:1.5 ratio on the surface of the T4MED sheet. Image analysis was used to measure the surface area covered. The increase in surface area showed there was a significant decrease in surface tension when the saliva was mixed with Cv3. The change in surface area was statistically significant and reflects a predicted decrease in surface tension of 40.9%.

Using the data shown in FIG. 14B, it was determined that the ratio of surface tension to inter feature distance for the Max Swab was 145.2 mN/m per mm for saliva only, which was sufficient to retain the saliva on the Max Swab without dripping off. However, the ratio of surface tension to inter feature distance for the Max Swab fell to 85.8 mN/m per mm for the saliva/preservative mixture, which allowed the sample to release from the swab. The ratio of liquid surface tension to the mean interspatial dimensions of swab features is referred to as the “Sample Retention Index”.

The Sample Retention Index for the different swab materials discussed above relative to FIG. 14A is set forth below in Table 1. Except as otherwise noted, the Sample Retention Index was determined for a saliva sample mixed with preservation reagent.

| | | Released |
|---|---|---|
| | Sample | sample without |
| | Retention Index | Centrifugation |
| Swab Material | (mN/m per mm) | (Yes/No) |
| T4MED Max Swab (saliva | 85.8 | Yes |
| with preservation reagent) | | |
| T4MED Max Swab (saliva only) | 145.2 | No |
| Foam Swab (100 ppi) | 171.6 | No |
| Cotton Swab | 3300 | No |
| Viscose Swab | 2145 | No |
| Polyester Spun Fiber Swab | 1129 | No |

As shown in Table 1, the Max Swab with only saliva had a Sample Retention Index that was lower than the Sample Retention Index for each of the other swabs with saliva mixed with preservation reagent. The Max Swab was able to reliably retain the biological sample without dripping, which could only be released via centrifugation. The Max Swab was also the only swab that was able to release saliva mixed with preservation reagent without centrifugation. The other swab materials retained the saliva/preservative mixture and required either expression (mechanical squeezing) or centrifugation to remove the sample. The other swab types had far higher sample retention indexes and failed to release the sample material (saliva with preservation solution) without centrifugation.

From this data one can reasonably conclude that a sample retention index of between 145.2 and 85.8 is the threshold for a sample to be passively released from a swab. The data also demonstrated that the design of the Max Swab was able selectively pass this threshold after the release of preservation solution from the cap into the sample preservation vessel. This represents a surprising feature that is distinct in a non-intuitive way even to someone skilled in the art. The data also suggest that a Sample Retention Index between about 100 mN/m per mm to about 5000 mN/m per mm, or about 120 mN/m per mm to about 4000 mN/m per mm, or about 140 mN/m per mm to about 3000 mN/m per mm, is adequate to retain a biological sample on the swab, and that a Sample Retention Index less than about 100 mN/m per mm, or less than about 95 mN/m per mm, or less than about 90 mN/m per mm, will allow the sample to be released without centrifugation after it is mixed with a preservation reagent.

### Definitions

Various aspects of the present devices and assemblies may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, assemblies, and methods may be illustrated with reference to one or more exemplary embodiments. As used herein, the terms “embodiment,” “alternative embodiment” and “exemplary embodiment” mean “serving as an example, instance, or illustration,” and should not necessarily be construed as required or as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. A sample collection swab for collecting a biological sample, comprising:
an elongated shaft extending from a first end to a second end along a longitudinal axis, wherein the elongate shaft is comprised of a flexible polymer; and
a swab body disposed at the first end of the elongated shaft, wherein the swab body is comprised of a flexible elastomer and includes a plurality of flexible protrusions extending laterally relative to the longitudinal axis and spaces between adjacent protrusions for collecting a biological sample;
wherein the swab body is more flexible than the elongated shaft.

2. The sample collection swab of claim 1, wherein the elongated shaft comprises a polyolefin.

3. The sample collection swab of claim 2, wherein the polyolefin is selected from polypropylene and polyethylene.

4. The sample collection swab of claim 1, wherein the elongated shaft has a thickness that decreases from the second end toward the first end.

5. The sample collection swab of claim 1, wherein the first end of the elongated shaft includes an enlarged connecting portion, and wherein the swab body is molded over the enlarged connecting portion.

6. The sample collection swab of claim 1, wherein the flexible polymer of the elongated shaft has a Shore A scale hardness in a range of about 50 to about 90, and wherein the flexible elastomer of the swab body has a Shore A scale hardness in a range of about 10 to about 70.

7. The sample collection swab of claim 1, wherein the swab body is comprised of a thermoplastic elastomer.

8. The sample collection swab of claim 7, wherein the thermoplastic elastomer is selected from thermoplastic vulcanizate (TPV) and thermoplastic polyurethane (TPU).

9. The sample collection swab of claim 8, wherein the thermoplastic vulcanizate (TPV) consists essentially of cured ethylene propylene diene monomer (EPDM) rubber particles encapsulated in a polypropylene (PP) matrix.

10. The sample collection swab of claim 1, wherein the swab body comprises a base having an encircling perimeter face extending between a first end and an opposing second end, the first end terminating at a proximal face that is coupled with the first end of the elongated shaft and the second end terminating at a distal face, the encircling perimeter face comprising opposing first and second side faces and opposing first and second end faces that each extend between the first end and the opposing second end of the base, wherein the plurality of flexible protrusions outwardly project from the first and second side faces.

11. The sample collection swab of claim 1, wherein the elongated shaft has a flexibility and the swab body has a flexibility so that, when used to collect a biological sample from a mucous membrane, the sample collection swab can collect mucus without significant physical removal of mucosal cells from the mucous membrane.

12. The sample collection swab of claim 1, further comprising a connection portion at the second end of the elongated shaft, wherein the connection portion is configured to associate with a handle, wherein the connection portion defines a shape complementary to a shape of an attachment portion of the handle.

13. The sample collection swab of claim 12, wherein the handle comprises a sealing cap configured to associate with a sample collection vessel.

14. The sample collection swab of claim 13, wherein the sealing cap comprises a reagent chamber for storing a preservation reagent.

15. The sample collection swab of claim 14, wherein the sealing cap is configured to release the preservation reagent from the reagent chamber upon associating the sealing cap with the sample collection vessel.

16. A sample collection system comprising:
the sample collection swab of claim 1;
a sealing cap attached to the second end of the elongated shaft of the sample collection swab, wherein the sealing cap comprises a reagent chamber for storing a preservation reagent; and
a sample preservation vessel defining a sample preservation chamber having an opening to receive the sample collection swab into the sample preservation chamber,
wherein the sealing cap is configured to release the preservation reagent from the reagent chamber upon associating the sealing cap with the sample collection vessel.

17. A sample collection system, comprising:
a sample collection swab, the sample collection swab comprising an elongated shaft extending along a longitudinal axis from a swab portion at a first end to a connection portion at a second end, the elongated body and the swab portion each being flexible;
a sample preservation vessel defining a sample preservation chamber having an opening to receive the sample collection swab into the sample preservation chamber; and
a sealing cap configured to associate with the sample preservation vessel and with the connection portion of the sample collection swab, wherein the sealing cap comprises a reagent chamber for storing a preservation reagent and a valve configured to release the preservation reagent from the reagent chamber and dispense it into the sample preservation chamber upon associating the sealing cap with the sample collection vessel.

18. The sample collection system of claim 17, wherein the valve comprises:
a core having one or more fluid channels or vents; and
a collar disposed around the core so as to occlude the one or more fluid channels or vents when in a closed configuration and not occlude the one or more fluid channels or vents when in an open configuration,
wherein associating the sealing cap with the sample preservation vessel causes a physical rearrangement of the core and the collar to move the valve from the closed configuration to the open configuration.

19. The sample collection system of claim 17, wherein the swab portion is configured and formed from an elastomer formulated so as to collect and retain mucus before placing the sample collection swab into the sample preservation chamber and thereafter release the mucus without centrifugation when mixed with the preservation reagent released from the sealing cap and dispensed into the sample preservation chamber.

20. The sample collection system of claim 19, wherein the swab portion has a Sample Retention Index in a range of about 100 mN/m per mm to about 5000 mN/m per mm when contacted with a biological sample comprising saliva or other mucus and a Sample Retention Index of less than about 100 mN/m per mm when the biological sample is mixed with the preservation reagent.

21. A method for collecting and preserving a biological sample, comprising:
providing a sample collection swab, the sample collection swab comprising an elongated shaft extending along a longitudinal axis from a swab portion at a first end to a connection portion at a second end, the elongated shaft and the swab portion each being flexible, wherein the connection portion is associated with a sealing cap, the sealing cap including a reagent chamber and a preservation reagent stored in the reagent chamber;
collecting a biological sample at a biological sample site by the swab portion of the sample collection swab;
inserting the sample collection swab into a sample preservation vessel; and
associating the sealing cap with the sample preservation vessel to enclose the sample collection swab and the biological sample therein and dispense the preservation reagent from the reagent chamber into the sample preservation vessel.

22. The method of claim 21, wherein the swab portion collects and retains the biological sample before placing the sample collection swab into the sample preservation vessel and thereafter releases the biological sample without centrifugation when mixed with the preservation reagent.
